(19) United States
(12) Patent Application Publication
(10) Pub. No.: US 2010/0298596 A1
(43) Pub. Date: Nov. 25, 2010

(54) PROCESS FOR THE PRODUCTION OF ISOCYANATES

(75) Inventors: Berthold Keggenhoff, Krefeld (DE); Heinrich Lokum, Kerpen (DE); Matthias Böhm, Leverkusen (DE)

Correspondence Address: BAYER MATERIAL SCIENCE LLC, 100 BAYER ROAD, PITTSBURGH, PA, 15205, US

(73) Assignee: Bayer MaterialScience AG, Leverkusen (DE)

(21) Appl. No.: 12/851,604

(22) Filed: Aug. 6, 2010

Related U.S. Application Data
(63) Continuation of application No. 11/801,636, filed on May 10, 2007, now abandoned.

(30) Foreign Application Priority Data
May 13, 2006 (DE) 102006022448.5

(51) Int. Cl. C07C 263/10

(52) U.S. Cl. 560/347

(57) ABSTRACT

Isocyanates, preferably diisocyanates and polyisocyanates of the diphenylmethane series (MDI), are produced by reaction of amines dissolved in a solvent with phosgene in the same solvent to form the corresponding isocyanates. Hydrogen chloride and excess phosgene are subsequently removed from the reaction mixture to obtain a crude isocyanate-containing solution. Subsequently, the crude isocyanate-containing solution is separated by distillation into isocyanates and solvent. The solvent is recycled and used for the production of solutions of the amines and of phosgene. The solvent being recycled is treated to reduce the phosgene and diisocyanate contents before being used for the production of the solution of the amine.

### BACKGROUND OF THE INVENTION

The present invention relates to a process for the production of isocyanates, preferably diisocyanates and polyisocyanates of the diphenylmethane series (MDI), by reacting amine dissolved in a solvent with phosgene to form the corresponding isocyanates, subsequent removal of hydrogen chloride and excess phosgene, subsequent separation by distillation of the crude solution containing the isocyanates obtained in this way into isocyanates and solvent, recirculation of the solvent and production of solutions of the amine and of phosgene, wherein the proportion of the solvent used for the production of the solution of the amine has low contents of phosgene and diisocyanate.

The production of isocyanates by reacting a primary amine with phosgene has been adequately known from the prior art for a relatively long time. A solution of the amine in a suitable solvent is generally reacted with a solution of phosgene in the same solvent. Processes for the production of organic isocyanates from a primary amine and phosgene are described in the literature, for example in Ullman's Encyclopedia of Industrial Chemistry, 5^{th} ed. vol. A 19, pp. 390 ff., VCH Verlagsgesellschaft mbH, Weinheim, 1991 and G. Oertel (ed.) Polyurethane Handbook, 2nd edition Hanser Verlag, Munich, 1993, pp. 60 ff., and G. Wegener et al. Applied Catalysis A: General 221 (2001), pp. 303-335, Elsevier Science B.V.

DE-A-19942299 describes a process for the production of mono- and oligoisocyanates by phosgenation of the corresponding amines in which a catalytic quantity of a monoisocyanate in an inert solvent is taken as an initial charge with phosgene. The amine, normally dissolved in solvent, is added and the reaction mixture obtained is reacted with phosgene. The process is comparatively complicated, due primarily to the use of the additional monoisocyanate which must later be separated off again. No teaching on the required purity of the solvent can be derived.

EP-A-1 073 628 describes a process for the production of mixtures of diphenylmethane diisocyanates and polyphenyl-polymethylene polyisocyanates (so-called polymeric MDI) by a two-step reaction of the mixture of the corresponding amines with phosgene in the presence of a solvent, maintaining selected ratios of phosgene and hydrogen chloride in the second process step. After the two-step reaction of the amine with phosgene in the selected solvent, the excess phosgene, the hydrogen chloride and the solvent are separated off from the reaction product (MDI) by distillation. EP-A-1 073 628 indicates that it is advantageous for good product quality if the residual content of phosgene in the reaction solution is <10 ppm after removal of the phosgene. Again, no teaching on the required purity of the circulating solvent can be derived.

Although it is not usually mentioned specifically in the literature of the prior art, it is generally known that the solvent that has been distilled off for the production of the amine and phosgene solution can be recirculated.

### SUMMARY OF THE INVENTION

The object of the present invention is to provide a process for the production of isocyanates using solvent recirculation, in which the formation of by-products and thus the losses of yield and quality impairment of the isocyanate produced are minimized.

It has now been found that the purity of the circulated solvent employed to produce the amine solution used in the phosgenation is of decisive importance for the formation of by-products in the crude isocyanate. Even a content of only 100 ppm phosgene or 100 ppm diisocyanate, based on the weight of the solvent, leads to detectable formation of by-products in the crude isocyanate. While this leads to a reduction in yield in the case of distilled isocyanates, i.e. in the isocyanates obtained as the overhead product, an undesirable influencing of the quality and of the reaction behavior is brought about in the isocyanates obtained as bottoms product (e.g., the diisocyanates and polyisocyanates of the diphenylmethane series) as a result. This can be detected, e.g., by chlorinated secondary components and an increased iron content.

It has also been found that the solvent recovered during the work-up and separation of the crude isocyanate solution contains several hundred ppm of free phosgene, based on the weight of the solvent. This is the case even when the crude isocyanate solution has previously been freed of phosgene to such an extent that no more free phosgene can be detected. Apparently, therefore, phosgene is formed or split off from secondary components during the work-up. Maintaining the phosgene content of the solvent to be recycled to the amine solution production step of the process of the present invention below is therefore a key feature of the present invention.

### BRIEF DESCRIPTION OF THE DRAWINGS

FIG. 1 is a diagram of the process according to the invention.

FIG. 2 is a diagram of the purification process by distillation of the solvent-containing stream.

FIG. 3 is a diagram of an alternative, particularly energy-efficient purification process by distillation of the solvent-containing stream.

### DETAILED DESCRIPTION OF THE PREFERRED EMBODIMENTS OF THE INVENTION

The present invention provides a process for the production of isocyanates by phosgenation of the corresponding amines in the presence of a solvent. In this process, a solution of amine in the solvent is produced and a solution of phosgene in the same solvent is produced. The solution of amine in the solvent and the solution of phosgene in the solvent are mixed together and the amine is reacted with the phosgene to form the corresponding isocyanate in an isocyanate-containing reaction solution. Hydrogen chloride and excess phosgene are separated off from the isocyanate-containing reaction solution thereby obtaining a crude isocyanate solution. The crude isocyanate solution is separated by distillation into an isocyanate-containing stream and a solvent-containing stream. At least a portion of the solvent-containing stream is recycled and used to produce the solution of amine in the solvent. The solvent-containing stream to be recycled is purified by distillation in such a way that the solvent-containing stream has a diisocyanate content of <100 ppm, preferably <50 ppm, most preferably <20 ppm, and a phosgene content of <100 ppm, preferably <50 ppm, most preferably <20 ppm, based in each case on the weight of the solvent-containing stream.

In principle, all primary amines with more than one primary amino group capable of reacting with phosgene to form one or more isocyanates with more than one isocyanate group are suitable as organic amines. Suitable amines have at least two, or optionally three or more, primary amino groups. The following are therefore suitable as organic primary amines: aliphatic, cycloaliphatic, aliphatic-aromatic, aromatic diamines and/or polyamines. Examples of suitable amines include: 1,4-diaminobutane, 1,6-diaminohexane, 1,8-diaminohexane, 1-amino-3,3,5-trimethyl-5-aminocyclohexane, lysine ethyl ester, lysine aminoethyl ester, 1,6,11-triaminoundecane, 1,5-naphthylenediamine, 1,4-diaminobenzene, p-xylylene-diamine, perhydrated 2,4- and/or 2,6-diaminotoluene, 2,2′-, 2,4′- and/or 4,4′-diaminodicyclohexylmethane, 2,4-, 2,6-diaminotoluene or mixtures thereof, 4,4′-, 2,4′- or 2,2′-diphenylmethanediamine or mixtures thereof, as well as higher molecular weight isomeric, oligomeric or polymeric derivatives of these amines and polyamines. Other possible amines are known from the prior art.

Preferred amines for the process of the present invention are the diamines and polyamines of the diphenylmethane series (MDA, monomeric, oligomeric and polymeric amines), technical mixtures of 2,4- and 2,6-diaminotoluene (TDA, toluenediamines) in a weight ratio of 80:20, isophoronediamine and hexamethylenediamine. Phosgenation produces the corresponding isocyanates, i.e., diisocyanatodiphenylmethane (MDI, monomeric, oligomeric and polymeric isocyanates), toluene diisocyanate (TDI), hexamethylene diisocyanate (HDI) and isophorone diisocyanate (IPDI). The process of the present invention is most preferably used for the production of the diisocyanates and polyisocyanates of the diphenylmethane series (MDI).

Solvents suitable for use in the process of the present invention include: chlorinated aromatic hydrocarbons, such as chlorobenzene, o-dichlorobenzene, p-dichlorobenzene, trichlorobenzenes, the corresponding chlorotoluenes or chloroxylenes, chloroethylbenzene, monochlorodiphenyl, α- or β-naphthyl chloride, ethyl benzoate, dialkyl phthalates, diisodiethyl phthalate, toluene and xylenes as well as methylene chloride, perchloroethylene, trichlorofluoromethane and/or butyl acetate. Mixtures of these solvents can also be used. Other examples of suitable, solvents are known from the prior art.

Chlorobenzene, dichlorobenzene and toluene are preferably used as solvents.

In a preferred embodiment of the process, the solvent-containing stream which is separated from the crude isocyanate solution and at least partly recycled is freed of residual quantities of phosgene in a special distillation step. During this separation of the residual quantities of phosgene by distillation, the perceived heat of the recovered solvent stream is most preferably used completely or partly as an energy source for this separation step. This can be done, for example, by using the feed into the distillation column to heat the bottom of the column by means of a heat exchanger. A suitable variant of this embodiment of the process of the present invention is illustrated in FIG. 3. Since the solvent separated off by distillation is normally obtained at a temperature of >100° C., while that used to produce the solution of amine in the solvent should be at a temperature of <50° C. for optimum phosgenation conditions, the separation of the residual quantities of phosgene can be associated with a simultaneous cooling of the solvent.

The isocyanate-containing stream preferably contains at least 95 wt. % of isocyanate, based on the weight of the isocyanate-containing stream. The solvent-containing stream preferably contains at least 95 wt. % of solvent, based on the weight of the solvent-containing stream.

The process of the present invention is explained in more detail below with reference to the Figures by way of example.

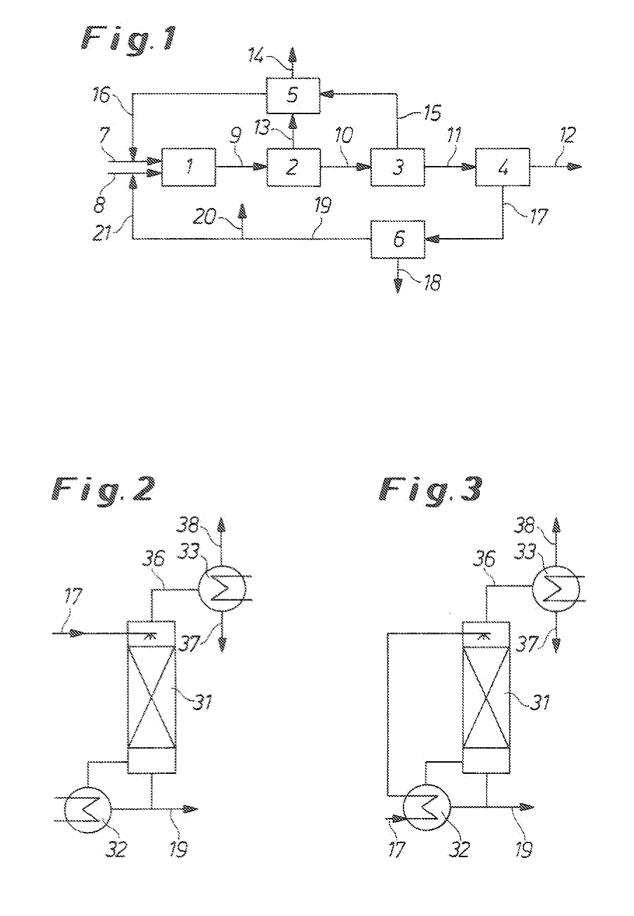

In FIG. 1, an example of the process of the present invention is illustrated diagrammatically.

In FIG. 1, Step 1 is the pre-phosgenation step in which the amine and phosgene solutions are mixed in mixer 1. In. Step 2, the hot phosgenation step, the amine and phosgene are reacted in phosgenation reactor 2. In Step 3, the dephosgenation step, hydrogen chloride and excess phosgene are separated off from the isocyanate-containing reaction solution by separation means 3. From an industrial point of view, it is preferred if a large part of the hydrogen chloride formed is already separated off directly at the outlet of the phosgenation reactor 2 together with the excess phosgene, and a further part in a dephosgenation column. In Step 4, the distillation step, the crude isocyanate solution obtained from dephosgenation Step 3 is worked up further and isocyanate and solvent are separated by distillation in distillation column 4. In Step 6, the solvent purification step, the purification by distillation of the solvent-containing stream obtained in Step 4 is purified by distillation in column 6 to separate off residual quantities of phosgene from the circulating solvent. In Step 5, the vapors obtained from Steps 2 and 3 are passed through vapor column 5 to recover phosgene and part of the solvent.

The solution of phosgene in the solvent (phosgene solution) is produced from fresh phosgene (stream 7) and recycled excess phosgene together with phosgene-containing solvent (stream 16). In parallel, the solution of amine in the solvent (amine solution) is produced from the amine (stream 8) and the recycled solvent stream (stream 21) largely freed of isocyanate and phosgene. It is, of course, also possible for one of the solutions to be produced at least partly with fresh solvent. The phosgene solution and the amine solution are reacted in the mixer 1, while mixing thoroughly, and the mixture thus obtained (stream 9) is reacted in the phosgenation reactor 2 by heating, with hydrogen chloride being split off, to form the isocyanate-containing reaction solution (stream 10). In the dephosgenation Step 3, the isocyanate-containing reaction solution is freed of residual quantities of phosgene by distillation and is fed into the distillation Step 4 as a practically phosgene-free crude isocyanate solution (stream 11). The vapors obtained in Steps 2 and 3, i.e. in the phosgenation reactor 2 and the dephosgenation Step 3 (streams 13 and 15), which consist substantially of hydrogen chloride, excess phosgene and portions of solvent, are separated in the vapor column 5 into hydrogen chloride (stream 14) and excess phosgene in solvent (stream 16). The hydrogen chloride (stream 14) is removed and preferably fed on for further utilization.

In the distillation Step 4, the crude isocyanate solution (stream 11) is separated by distillation into the isocyanate (isocyanate-containing stream 12) and the recovered solvent (solvent-containing stream 17). Since the isocyanate normally has a higher boiling point than the solvent, it can be ensured by suitable design of the work-up in the distillation Step 4 that the solvent (solvent-containing stream 17) possesses the required low diisocyanate content of <100 ppm, preferably <50 ppm, most preferably <20 ppm, based on the weight of the solvent-containing stream.

However, since phosgene is split back from secondary components of the phosgenation during the work-up in the distillation Step 4, the solvent-containing stream (stream 17) always has a residual phosgene content. This is now separated off in the solvent purification system 6 as a phosgene-enriched solvent stream (stream 18) and can be fed back into the process and added to the stream 16, for example (not illustrated in FIG. 1). The purified solvent-containing stream 19 with a phosgene content of <100 ppm, preferably <50 ppm, most preferably <20 ppm, and a diisocyanate content of <100 ppm, preferably <50 ppm, most preferably <20 ppm, based on the weight of the solvent-containing stream in each case, can be partly removed as stream 20 and used at another point in the process, but is at least partly, preferably predominantly, employed as stream 21 for the production of the amine solution.

The reaction of the amine solution with the phosgene solution in Steps 1 and 2 generally takes place at temperatures of from 20 to 240° C. and under absolute pressures of 1 to 50 bar. It can be carried out in one or more steps, phosgene generally being employed in a stoichiometric excess. In Step 1, the amine solution and the phosgene solution are combined, preferably using static mixing elements or special dynamic mixing elements, and then passed in Step 2, e.g., from bottom to top through one or more reaction towers in which the mixture reacts to form the desired isocyanate. In addition to reaction towers, which are provided with suitable mixing elements, reaction vessels with an agitator device can also be used. Suitable static and dynamic mixing elements and reaction equipment are known from the prior art.

The separation of residual phosgene and hydrogen chloride from the isocyanate-containing reaction solution obtained takes place advantageously in the dephosgenation Step 3, the isocyanate-containing reaction solution being fed into the stripping section of a distillation column. This distillation step is preferably carried out in such a way that the dephosgenated crude isocyanate solution is obtained as a bottoms product with a residual phosgene, content of <100 ppm, preferably <10 ppm, based on the weight of the crude isocyanate solution.

The separation of the crude isocyanate solution by distillation takes place in a manner adapted to the respective boiling points of solvent and isocyanate in a one-step or preferably multi-step distillation sequence in the distillation Step 4. Distillation sequences of this type are known from the prior art and described, e.g., for TDI in EP-A 1371633 and EP-A 1371634.

In the preferred case of the production of MDI using monochlorobenzene as the solvent, this separation by distillation can advantageously take place in such a way that the crude isocyanate solution is worked up in two steps into a bottoms product containing at least 95 wt. %, most preferably at least 97 wt. %, of isocyanate, based on the weight of the isocyanate-containing stream, which is preferably then freed of low boiling materials in additional steps. In the first step, 60-90% of the solvent contained in the crude isocyanate solution is preferably separated off by a flash distillation under absolute pressures of from 600-1200 mbar and at bottom temperatures of from 110-170° C., the vapors being worked up in a distillation column with 5-20 separation stages and 10-30% reflux, so that a solvent-containing stream with a diisocyanate content of <100 ppm, preferably <50 ppm, most preferably <20 ppm, based on the weight of the solvent-containing stream, is achieved. In the second step, the residual solvent is separated off to a residual content of 1-3 wt. % in the bottoms product under absolute pressures of 60-140 mbar and at bottom temperatures of from 130-190° C. The vapors can also be worked up in a distillation column with 5-20 separation stages and 10-40% reflux, so that a solvent-containing stream with a diisocyanate content of <100 ppm, preferably <50 ppm, most preferably <20 ppm, based on the weight of the solvent-containing stream, is achieved, or they can be recirculated back into the first distillation step as feed after condensation. In the same way, the distillate streams separated off in the following steps can be recirculated back into the first distillation step as feed.

In this way, the entire solvent-containing stream can be separated off advantageously with the required specification regarding diisocyanate (<100 ppm of diisocyanates, based on the weight of the solvent-containing stream). This solvent-containing stream can, however, contain monoisocyanates (e.g., phenyl isocyanate) as an impurity with a content of 100-1000 ppm and a residual phosgene quantity of 100-1000 ppm.

Even though it is possible, in principle, to design the distillation step so that the solvent-containing stream is taken off (e.g., as a side stream) from a column in a quality that meets the required specification both regarding diisocyanate content and regarding phosgene content (<100 ppm of diisocyanates and <100 ppm of phosgene, based in each case on the weight of the solvent-containing stream), it is generally more favorable to design this distillation only according to the diisocyanate content to be achieved of <100 ppm, preferably <50 ppm, most preferably <20 ppm, and to remove the residual phosgene content, which is then usually 100-1000 ppm, in the separate Step 6.

One possible version of the solvent purification by distillation in Step 6 is illustrated in FIG. 2. The solvent purification system includes a stripper column 31, a bottom evaporator 32 and an overhead condenser 33. The-solvent-containing stream 17 from the work-up in Step 4 having a low phosgene content (not illustrated in FIG. 2) is fed into the stripper column 31, which preferably has 4-20 separation stages. The bottom evaporator 32 produces sufficient quantities of vapors, by heating (e.g., with heating steam) so that the dephosgenated solvent-containing stream 19 now only possesses a phosgene content of <100 ppm, preferably <50 ppm, most preferably <20 ppm, and a diisocyanate content of <100 ppm, preferably <50 ppm, most preferably <20 ppm, based in each case on the weight of the solvent-containing stream, and can thus be used to produce the amine solution. The vapor stream 36 produced contains the separated phosgene at preferably 1-6 wt. %, based on the weight of the vapor stream, and is preferably condensed in the condenser 33; while the condensate 37 is fed into the isocyanate process (e.g., to prepare the phosgene solution) and the residual gases 38 are preferably fed to the waste gas work-up. However, the condensate 37 can also be completely or partly recirculated as reflux to the stripper column 31, as a result of which the phosgene becomes further concentrated in the vapor stream 36. If Step 6 is operated under a pressure below the boiling point of the solvent in the solvent-containing stream 17, partial separation of phosgene already occurs at the entrance to the stripper column 31 by flashing out. Thus, the amount of energy to be fed into the evaporator 32 is reduced.

FIG. 3 shows an embodiment of the solvent purification by distillation in Step 6 that is particularly preferred because it is particularly energy-efficient.

The solvent-containing stream 17 from the work-up in Step 4 having a low phosgene content (not illustrated in FIG. 3) is first fed as a heating agent through the bottom evaporator 32 and then into the stripper column 31, which has 4-20 separation stages. The bottom evaporator 32 produces sufficient quantities of vapors, as a result of being heated with the solvent-containing stream, so that the dephosgenated solvent-containing stream 19 now only possesses a phosgene content of <100 ppm, preferably <50 ppm, most preferably <20 ppm; and a diisocyanate content of <100 ppm, preferably <50 ppm, most preferably <20 ppm, based in each case on the weight of the solvent-containing stream, and can thus be used to produce the amine solution. During this process, the solvent stream is cooled by 2-10° C. The vapor stream 36 produced contains the separated phosgene at preferably 1-6 wt. %, based on the weight of the vapor stream, and is preferably condensed in the condenser 33; while the condensate 37 is fed into the isocyanate process (e.g., to prepare the phosgene solution) and the residual gases 38 are preferably fed via a vacuum system to the waste gas work-up. However, the condensate 37 can also be completely or partly recirculated into the stripper column 31 as reflux, as a result of which the phosgene becomes further concentrated in the vapor stream 36. By regulating the pressure in the system, the quantity of vapors produced, and thus the quality or purity of the solvent-containing stream, is regulated. Overall, the separation of the residual quantities of phosgene is achieved without any external energy input, which even brings about, at the same time, a generally desirable cooling of the solvent-containing stream, which is used to produce the amine solution.

### EXAMPLES

### Example 1

### Production of a Mixture of Diamines and Polyamines

In a stirred vessel, 2600 g aniline were thoroughly mixed with 1000 g formalin (30 wt. % aqueous solution of formaldehyde, based on the weight of the solution) at 25° C., with stirring, during which the mixture heated up to 60° C. The stirrer was turned off and the aqueous phase settling out at the top was separated off 68 g of 30 wt. % aqueous hydrochloric acid, were then mixed in, while stirring again and cooling, maintaining a temperature of 45° C. After continuing to stir at this temperature for 15 min, the cooling was replaced by heating and the mixture was uniformly heated to 140° C. in the course of 120 min under a pressure of 5 bar, and was then kept at this temperature for 15 min.

The mixture was then cooled to 100° C., depressurized to normal pressure and neutralized by adding 54 g of 50 wt. % aqueous sodium hydroxide solution while stirring. After turning off the stirrer, the phases were allowed to settle and the aqueous phase at the bottom was sucked off. Excess aniline was then distilled off with residual water that remained, initially under normal pressure, and the aniline residues were removed by incipient distillation, at 100 mbar and 250° C., of the polyamine mixture obtained.

1900 g of a mixture of diamines and polyamines of the following composition were obtained:

4,4′-MDA: 60.1 wt. %

2,4′-MDA: 6.0 wt. %

2,2′-MDA: 0.2 wt. %

higher molecular weight polyamines: 33.7 wt. %; based in each case on the weight of the mixture.

### Example 2

### Production of a Mixture of Diisocyanates and Polyisocyanates Using Contaminated Solvent (Not According to the Invention)

In a stirred reactor, 1900 g of the mixture of diamines and polyamines obtained in Example 1 were dissolved in 5700 g chlorobenzene with a content of 200 ppm phosgene and 200 ppm MDI, based in each case on the weight of the solvent chlorobenzene. In a second vessel made of stainless steel (DIN 1.4571), a 33 wt. % (based on the weight of the solution) phosgene solution was prepared by dissolving 3800 g phosgene in 7600 g chlorobenzene while cooling to 0° C., and the amine and phosgene solutions were mixed while stirring intensively. The resulting suspension of solids was then heated slowly with the formation of hydrogen chloride gas, which was withdrawn by suitable means. During this process, a homogeneous solution of the polyisocyanate was formed. The solvent was now separated off by distillation, as a result of which 2370 g of a mixture of diisocyanates and polyisocyanates of the following composition was obtained:

4,4′-MDI: 59.2 wt. %

2,4′-MDI: 5.4 wt. %

2,2′-MDI: 0.2 wt. %

higher molecular weight polyisocyanates: 35.2 wt. %, based in each case on the weight of the mixture.

Acidity (ASTM D 1638-74): 180 ppm

Iron content: 10 ppm

Extinction of a 2% solution in chlorobenzene (wavelength 430 nm, film thickness 10 mm): 0.27

### Example 3

### Production of a Mixture of Diisocyanates and Polyisocyanates Using Pure Solvent (According to the Invention)

In a stirred reactor, 1900 g of the mixture of diamines and polyamines obtained in Example 1 were dissolved in 5700 g chlorobenzene with a content of 20 ppm phosgene and 20 ppm MDI, based in each case on the weight of the solvent chlorobenzene. In a second vessel made of stainless steel (DIN 1.4571), a 33 wt. % (based on the weight of the solution) phosgene solution was prepared by dissolving 3800 g phosgene in 7600 g chlorobenzene while cooling to 0° C., and the amine and phosgene solutions were mixed into this while stirring intensively. The resulting suspension of solids was then heated slowly with the formation of hydrogen chloride gas, which was withdrawn by suitable means. During this process, a homogeneous solution of the polyisocyanate was formed. The solvent was then separated off by distillation, as a result of which 2370 g of a mixture of diisocyanates and polyisocyanates of the following composition was obtained:

4,4′-MDI: 59.3 wt. %

2,4′-MDI: 5.5 wt. %

2,2′-MDI: 02 wt. %

higher molecular weight polyisocyanates: 35 wt. %, based in each case on the weight of the mixture.

Acidity (ASTM D 1638-74): 62 ppm

Iron content: 4 ppm

Extinction of a 2% solution in chlorobenzene (wavelength 430 nm, film thickness 10 mm): 0.13

Thus, when the results of Examples 2 and 3 are compared, it is shown that the use of purified solvent for the production of the amine solution in accordance with the process of the present invention results in an isocyanate being obtained with improved quality, which is expressed as a low acidity, a low iron content and a light color (low extinction).

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

1. A process for the production of diisocyanates and polyisocyanates of diphenylmethane (MDI) comprising:
a) producing a solution of a diamine and/or polyamine of diphenylmethane in a solvent,
b) producing a solution of phosgene in the same solvent used to produce the amine solution of a), and
c) combining the solution of amine produced in a) and the solution of phosgene produced in b),
d) reacting the amine in the solution of amine produced in a) with the phosgene in the solution of phosgene produced in b) to form an MDI-containing reaction solution,
e) separating hydrogen chloride and excess phosgene from the MDI-containing reaction solution to obtain a crude MDI solution,
f) distilling the crude MDI solution to separate the crude MDI solution into
(1) an MDI-containing stream and
(2) a solvent-containing stream containing from 100 to 1000 ppm of residual phosgene and less than 100 ppm MDI,
g) purifying the solvent-containing stream (2) from f) in a stripper column to obtain a purified solvent-containing stream having a diisocyanate content of <100 ppm and a phosgene content of <100 ppm, based in each case on the weight of the purified solvent-containing stream, and
h) recycling at least a portion of the solvent-containing stream from g) into step a).

2. The process of claim 1, in which a portion of the purified solvent-containing stream is recycled to step b) to be used to produce the solution of phosgene in the solvent.

3. (canceled)

4. The process of claim 1 in which the solvent is chlorobenzene, dichlorobenzene and/or toluene.

5. The process of claim 1 in which the-solvent-containing stream purified in step g) is obtained as a bottoms product of the stripper column and the solvent-containing stream (2) from f) is cooled by heat exchange with the bottom of the stripper column.
